(19) United States
(12) Patent Application Publication
(10) Pub. No.: US 2015/0204872 A1
(43) Pub. Date: Jul. 23, 2015

(54) Method of Determining the Quantity of Enzyme Complex Components

(71) Applicant: PharmaCadence Analytical Services, LLC, Hatfield, PA (US)

(72) Inventors: Richard King, Quakertown, PA (US); Victoria King, Quakertown, PA (US); Carmen Fernandez-Metzler, Doylestown, PA (US); Bonnie Baker, Warrington, PA (US)

(73) Assignee: PharmaCadence Analytical Services, LLC, Hatfield, PA (US)

(21) Appl. No.: 14/162,084

(22) Filed: Jan. 23, 2014

(51) Int. Cl. G01N 33/573

(52) U.S. Cl. CPC G01N 33/573

(57) ABSTRACT

Provided herein are new methods for determining the quantity of related enzyme complex components present in biological samples. The target analytes residing in the same original sample are related as components of a single enzyme complex or multiple enzyme complexes and are analyzed by the same analytical technique from the same original sample which may be split, fractionated, or analyzed directly.

### STATEMENT REGARDING FEDERALLY SPONSORED RESEARCH OR DEVELOPMENT

The present invention was not developed with the use of any Federal Funds, but was developed independently by the inventor.

### TECHNICAL FIELD OF THE INVENTION

The present invention relates generally to the field of analytical chemistry, biochemistry, and molecular diagnostics, and particularly to the method of performing quantitative analysis of proteins and related molecules responsible for biochemical actions.

### BACKGROUND

Scientific and medical research begins with the attempt to isolate components of interest. The field of research is replete with references and technologies that aim to detect certain specific proteins or molecules. Traditional analysis of biological systems involves separate analysis of the proteins and small molecules that work in concert to create a biochemical pathway. Homogeneous binding tests are well known for quantitative or qualitative detection of an analyte in an assay using suitable reagents, such as for example, which disclosed in U.S. Pat. No. 8,399,209 B2 to Schelp et al. Also commonly practiced in the art is screening by immobilization on a substrate. U.S. patent application Ser. No. 11/375,054 to Brook et al. discloses screening for protein modulators by contacting stream having substrate with a monolithic chromatographic stationary phase to produce product, introducing the test compounds into a stream, and observing the conversion of a substrate to product by the protein and determining a product to substrate ratio. Changes in this ratio in the presence of a certain compound indicates that such compound is a modulator of the enzyme. U.S. patent application Ser. No. 10/333,734 to Martin Glennsbjerg discloses a spatially resolved enzyme-linked assay. Disclosed is a method for assessing the quality and quantity of analyte in a sample which involves arranging a catalyst-analyte complex between the analyte and the catalyst in the sample domain, contacting the substrate, recording the image of the product and correlating the data.

A continuing and unmet need exists for new and improved methods to perform quantitative analysis of proteins and related small molecules responsible for biochemical actions. It is advantageous to analyze these components in a fashion analogous to the way they work naturally. Instead of separating the different chemotypes into completely separate analytical workflows, a method whereby the proteins and small molecules can be analyzed from the same samples in a concerted workflow based on analysis of the enzyme complex components would lead to simplification and efficiency of quantitative analysis.

The invention disclosed herein is a method to determine the quantity of related enzyme complex components in the same analytical sample. Prior to the invention herein, small molecules and proteins were analyzed in separate samples if not separate experiments. The practice derives from the specialized procedures needed to isolate, treat, and detect the different types of molecules (proteins and small molecules). From that basic premise, entire workflows were developed for the dedicated determination of small molecules and proteins as separate processes. While sample volume is often sufficient to split samples into different aliquots that can be taken through separate processes, the storage, treatment, and handling of the separate aliquots often introduces unaccounted for artifacts into the results. These unintended, perhaps unknown, systematic errors make recombination of the data from split samples widely variable at best and uninterpretable at the extreme.

By measuring the complete set of enzyme complex components, more information can be obtained relating to the activity and action of the enzyme system than is possible with current methods. Importantly, variability introduced by separating the experiments and analyses traditionally conducted to understand and monitor enzyme system function can be removed or dramatically reduced by analyzing the entire enzyme complex within the same samples.

Efforts to integrate the analysis of proteins and small molecules for untargeted search or the integration of proteomics and metabolomics are beginning to be investigated as a more relevant way to identify biomarkers. Rather than look for a single molecule as a marker, the hypothesis maintains that more complete system function will provide new and useful markers for relevant biological changes of consequence. Samples are often split for analysis by different techniques.

This is a relatively common practice in analytical and clinical chemistry. Also, samples are fractionated and the fractions investigated by different techniques. Environmental testing routinely analyzes acid and base-neutral fractions from a single original sample for multiple small molecule analytes.

The more practical and commercially valuable extension of this hypothesis is the measurement of targeted enzyme complex components known or suspected to be related to the functional enzyme. As an example, the concentration of substrate, enzyme, and product give a more complete picture of the functional enzyme complex than the measurement of any single chemical component. Additionally, the measurement of all three from the same sample provides more relevant information than data collected on individual components in separate assays or even separate experiments.

In order to make such measurements possible, the invention herein provides a method that permits large proteins to be quantified in the same samples from which small molecules are quantified. Such a method could enable the development and practical use of a new generation of biomarkers based on collections of related molecules rather than a single compound. The pattern of change observed with experimental conditions for the collection of molecules, the enzyme complex, may hold diagnostic information unattainable by the same pattern of a single compound. Additionally, the relevant changes in the patterns may be more obvious when the enzyme complex is measured in a process that works from the same physical sample in the same workflow.

### SUMMARY

Provided herein are methods for determining the quantity of related enzyme complex components present in biological samples. The target analytes residing in the same original sample are related as components of a single enzyme complex or multiple enzyme complexes and are analyzed by a single analytical procedure from the same original sample which may be split, fractionated, or analyzed directly.

### BRIEF DESCRIPTION OF THE DRAWINGS

FIG. 1 is a schematic representation of the enzyme complex for the UGT 2B7 enzyme.

FIG. 2 is a schematic representation of a typical workflow for the analysis of small molecules from a biological matrix sample.

FIG. 3 is a schematic representation of a typical workflow for the analysis of proteins from a biological matrix sample.

FIG. 4 is a schematic representation of the method of the invention showing the workflow for determining the quantity of enzyme complex components from a single original sample.

FIG. 5 is a graph showing the detector response versus chromatographic retention time for each of the measured enzyme complex components from a single sample run in a concerted analytical method as described in Example 1.

FIG. 6 is a graph showing an embodiment of the invention as described in Example 1.

FIG. 7 is a graph of the results of Example 2 showing the increasing formation of the enzyme complex product, AZT-glucuronide with increasing amount of recombinant microsomal protein added to an activity experiment.

FIG. 8 is a graph of the results of Example 2 showing the consumption of the enzyme complex substrate, AZT, with increasing amount of recombinant microsomal protein added to an activity experiment.

FIG. 9 is a graph of results showing the increase in measured UGT2B7 protein found upon addition of recombinant microsomes over-expressing the UGT 2B7 protein to an enzyme activity experiment.

### DETAILED DESCRIPTION OF THE INVENTION

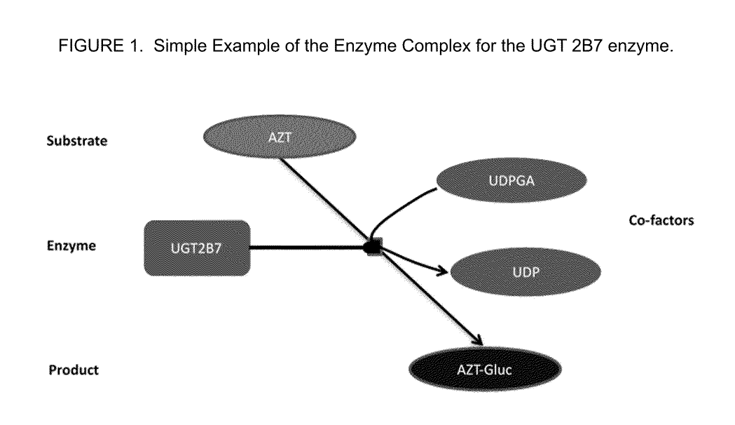

As used herein, the term “enzyme complex component” includes the proteins involved with the action of the enzyme itself. These proteins include the enzyme and any other proteins required for enzyme activity. By “proteins” is meant polypeptides, antibodies, peptides and other fragments or components commonly referred to as proteins. The enzyme complex components also include small molecules that interact with the enzyme in different roles including, but not limited to, substrate, metabolite, inhibitor, activator, co-enzyme and co-factor. As used herein, small molecules” refers to molecules with a molecular weight less than 2000 Daltons. FIG. 1 shows the basic concept of the enzyme complex as used herein.

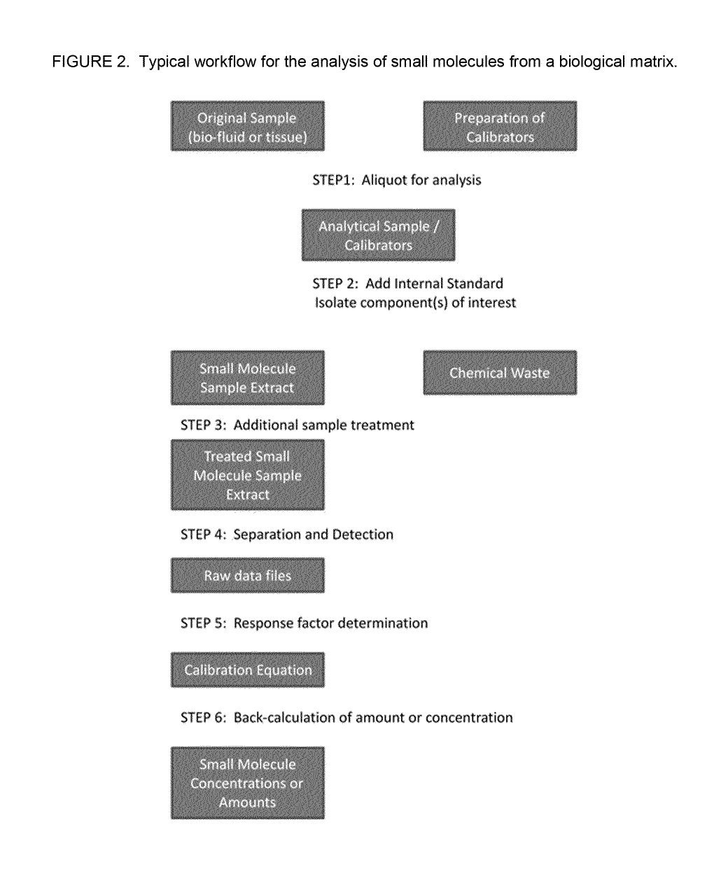

FIG. 2 demonstrates a typical workflow for the analysis of small molecules from a biological matrix. The diagram shows the steps and procedures used to extract small molecules from biological matrices including but not limited to blood, plasma, serum, cerebrospinal fluid (CSF), tissue homogenates, cells, cell lysates bile, and urine. In Step 1, a portion or aliquot of the original sample is exactly measured into a clean vessel becoming the analytical sample. In Step 2, the analytical internal standard may be but is not required to be added to the analytical sample and the analytical sample is simplified by means of removing unwanted sample components or by attempting to isolate the component(s) of interest. The portion of the sample containing the small molecules to be analyzed is the extract. The remainder is chemical waste or mixed chemical waste containing the remaining sample components to be properly discarded. In Step 3, the extract might be further treated to make it amenable to analysis. Processes such as filtration, evaporation and reconstitution, dilution and derivatization are examples of extract treatment that are typically used. In Step 4, the treated sample extract or simplified sample is subjected to further separation of components and detection of those components. In Step 5, the response factor of the detection method is calibrated using calibrators of known concentration. In Step 6, the quantity of the analyte is determined by the detection response and the calibrated response factor.

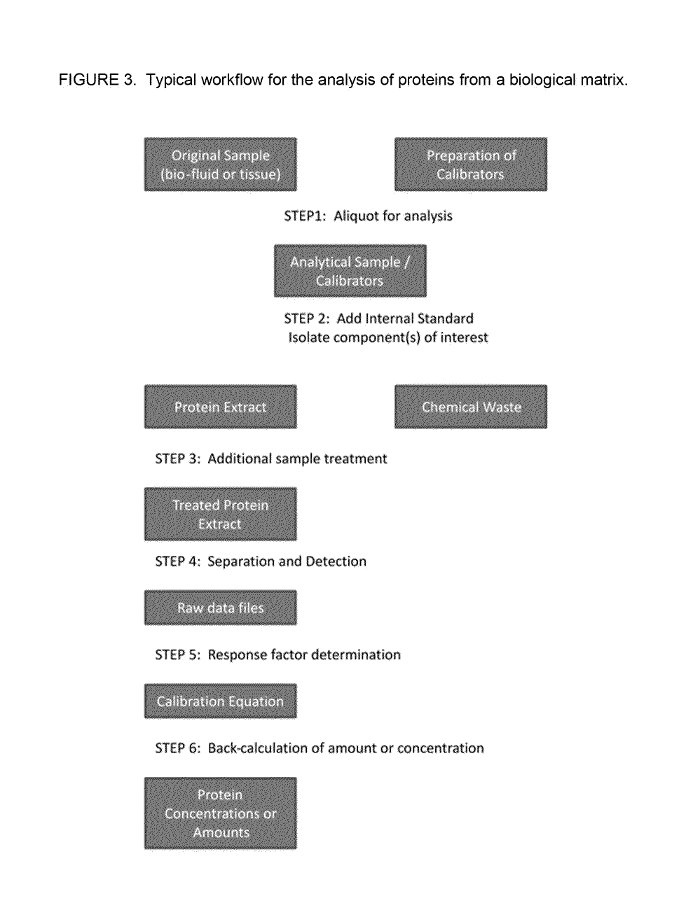

FIG. 3 demonstrates the typical workflow for the analysis of proteins from a biological matrix. The diagram shows the steps and procedures used to extract proteins and large peptides from biological matrices including but not limited to blood, plasma, serum, cerebrospinal fluid (CSF), tissue homogenates, cells, cell lysates, bile, and urine. In Step 1, a portion or aliquot of the original sample is measured precisely into a clean vessel becoming the analytical sample. In Step 2, the sample may be simplified by means of removing unwanted sample components or by attempting to isolate the component of interest. The portion of the sample containing the proteins to be analyzed is the extract. The remainder is chemical waste, biological waste or mixed chemical waste containing the remaining sample components to be properly discarded. In Step 3, the extract might be further treated to make it amenable to analysis. Processes such as filtration, evaporation and reconstitution, dilution, chemical or biological digestion and derivatization are examples of extract treatment that are typically used. In Step 4, the treated sample extract or simplified sample is subjected to further separation of components and detection of those components. In Step 5, the response factor of the detection method is calibrated using calibrators of known concentration. Finally, in Step 6, the quantity of the analyte is determined by the detection response and the calibrated response factor.

According to the method of the invention, an extract is derived from a biological matrix. The biological matrix can be a natural fluid derived from an animal, such as but not limited to blood, plasma, serum, cerebrospinal fluid, bile, or urine; or a natural fluid derived from a plant, such as, for example, from the juice, sap, or oil of a plant. In preferred embodiments, the biological matrix is a tissue, a tissue homogenate, a cell, a cell lysate, or a fraction thereof.

The biological matrix typically contains large molecules and small molecules, including enzyme complex components. The method comprises digesting the large molecule proteins of the biological matrix sample into peptides using a chemical or enzymatic method, optionally, contacting the biological matrix sample with a solution having a pH less than 5.0 or greater than 8.0, an organic solvent or at least one affinity reagent to form a precipitate, optionally, separating the supernatant from the precipitate by centrifugation or filtration, optionally, treating the precipitate by resuspension in a solution having a pH in the range of 6.8 and 8.0, heating said solution to a temperature above to 50° C. or above, reducing said solution with at least one chemical reducing agent, alkylating said solution, optionally, digesting the proteins present in the precipitate into peptides using a chemical or enzymatic method, determining the quantity of the enzyme complex related small molecules, and determining the quantity of the enzyme complex related peptides obtained.

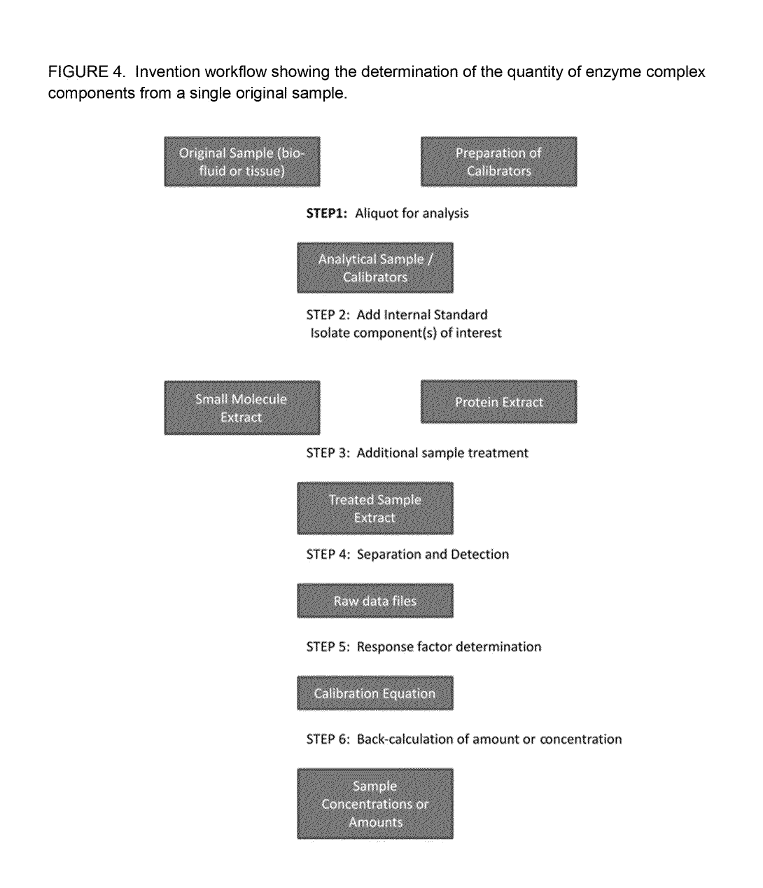

FIG. 4 illustrates the preferred embodiment of the invention. The schematic workflow shows the determination of the quantity of enzyme complex components from a single original sample. In Step 1, a portion or aliquot of the original sample is measured exactly into a clean vessel becoming the analytical sample. In Step 2, the sample may be simplified by means of removing unwanted sample components or by attempting to isolate the components of interest. The portion of the sample that contains the small molecules and proteins to be analyzed is the extract. The remainder is chemical waste, biological waste or mixed chemical waste to be properly discarded. In Step 3, the extract might be further treated to make it amenable to analysis. Processes such as filtration, evaporation and reconstitution, dilution, chemical or biological digestion and derivatization are examples of extract treatment that might be applied to the small molecules in the extract while processes such as filtration, evaporation and reconstitution, dilution, chemical or biological digestion and derivatization are examples of extract treatment that might be applied to the proteins in the extract. In Step 4, the treated sample extract or simplified sample is subjected to further separation of components and detection of those components. In Step 5, the response factor of the detection method is calibrated using calibrators of known concentration. In Step 6, the quantity of the analyte is determined by the detection response and the calibrated response factor.

In the preferred embodiment, a sample containing the protein complex of interest is digested into peptides by chemical or enzymatic digestion with or without prior reduction and alkylation or other treatment of the proteins in the sample prior to digestion. The resulting mixture of polypeptides and small molecules is analyzed to determine the quantity of target enzyme complex components present in the sample. The analysis of multiple different chemical types or chemotypes in a single sample will be referred to as chemoplexing.

In another embodiment of the invention, a sample containing the protein complex of interest is treated by the addition of an organic solvent such as acetonitrile to precipitate the large proteins and facilitate the analysis of the small molecules. The supernatant from the protein precipitation is removed from the protein pellet and analyzed for the quantity of enzyme complex related small molecules including but not limited to substrate, metabolite, inhibitor, activator, co-enzyme and co-factor. The remaining protein pellet is digested into peptides by chemical or enzymatic digestion with or without prior reduction and alkylation or other treatment of the protein prior to digestion. The resulting peptide mixture is analyzed to determine the quantity of target enzyme and/or co-enzyme present in the sample. The quantity of both small molecules and large molecules associated with the enzyme complex is then determined from the same original sample aliquot.

In another embodiment of the invention, a sample containing the protein complex of interest is treated by selected removal of the large molecules using a molecular weight cutoff membrane. The membrane may effect separation by means of dialysis mechanisms, vacuum filtration mechanisms, centrifugation or other application of forces that create a separation across the membrane of the protein and small molecules associated with the enzyme complex. The portion of the sample containing the small molecules is analyzed for the quantity of enzyme complex related small molecules including but not limited to substrate, metabolite, inhibitor, activator, and co-factor. The portion containing the protein is digested into peptides by chemical or enzymatic digestion with or without prior reduction and alkylation or other treatment of the protein prior to digestion. The resulting peptide mixture is analyzed to determine the quantity of target enzyme and/or co-enzyme present in the sample.

In another embodiment of the invention, a sample containing the protein complex of interest is treated by selected removal of the large molecules using size exclusion mechanisms found in gel filtration chromatography, gel electrophoresis, size exclusion chromatography and gel permeation chromatography. The portion of the sample containing the small molecules is analyzed for the quantity of enzyme complex related small molecules including but not limited to substrate, metabolite, inhibitor, activator, co-enzyme and co-factor. The portion containing the protein is digested into peptides by chemical or enzymatic digestion with or without prior reduction and alkylation or other treatment of the protein prior to digestion. The resulting peptide mixture is analyzed to determine the quantity of target enzyme and/or co-enzyme present in the sample.

In another embodiment of the invention, a sample containing the protein complex of interest is treated by selected removal of the large molecules using centrifugation in a density gradient medium. The portion of the sample containing the small molecules is analyzed for the quantity of enzyme complex related small molecules including but not limited to substrate, metabolite, inhibitor, activator, co-enzyme and co-factor. The portion containing the protein is digested into peptides by chemical or enzymatic digestion with or without prior reduction and alkylation or other treatment of the protein prior to digestion. The resulting peptide mixture is analyzed to determine the quantity of target enzyme and/or co-enzyme present in the sample.

In another embodiment of the invention, a sample containing the protein complex of interest is treated by selected removal of the large molecules using a solid phase extraction procedure (SPE). Solid phase extraction may effect separation by means of ion exchange, reverse phase, mixed mode, hydrophilic interaction liquid chromatography (HILIC), and/or normal phase mechanisms that create a separation across the solid phase containing surface or volume of the protein and small molecules associated with the enzyme complex.

The portion of the sample containing the small molecules is analyzed for the quantity of enzyme complex related small molecules including but not limited to substrate, metabolite, inhibitor, activator, co-enzyme and co-factor. The portion containing the protein is digested into peptides by chemical or enzymatic digestion with or without prior reduction and alkylation or other treatment of the protein prior to digestion. The resulting peptide mixture is analyzed to determine the quantity of target enzyme and/or co-enzyme present in the sample.

In another embodiment of the invention, a sample containing the protein complex of interest is treated by selected removal of the large molecules using affinity binding. The portion of the sample containing the small molecules is analyzed for the quantity of enzyme complex related small molecules including but not limited to substrate, metabolite, inhibitor, activator, co-enzyme and co-factor. The portion containing the protein and binding agent is digested into peptides by chemical or enzymatic digestion with or without prior reduction and alkylation or other treatment of the protein prior to digestion. The resulting peptide mixture is analyzed to determine the quantity of target enzyme and/or co-enzyme present in the sample.

In another embodiment of the invention, a sample containing the protein complex of interest is treated by selected removal of the small molecules using affinity binding. The portion of the sample containing the small molecules together with the affinity agent is analyzed for the quantity of enzyme complex related small molecules including but not limited to substrate, metabolite, inhibitor, activator, co-enzyme and co-factor. The portion containing the protein and binding agent is digested into peptides by chemical or enzymatic digestion with or without prior reduction and alkylation or other treatment of the protein prior to digestion. The resulting peptide mixture is analyzed to determine the quantity of target enzyme and/or co-enzyme present in the sample.

In another embodiment of the invention, a sample containing the protein complex of interest is treated by selected removal of the large molecules and small molecules using affinity binding. Affinity binding agents that selectively bind the enzyme complex small molecules can be mixed with affinity agents that selectively bind the protein enzyme complex components creating a mixture of affinity agents selective for the components of the enzyme complex. The resulting mixture of large and small molecules associated with the enzyme complex may be subjected to one or several of the previous embodiments of the invention for the analysis of the enzyme complex components from a single sample aliquot.

In another embodiment of the invention, a sample containing the protein complex of interest is digested into peptides by chemical or enzymatic digestion with or without prior reduction and alkylation or other treatment of the protein prior to digestion. The resulting peptide mixture is analyzed to determine the quantity of target enzyme and/or co-enzyme present in the sample. The small molecule enzyme complex components are also determined from this sample.

Any or all of these embodiments may be applied to mixtures of enzyme complexes permitting the determination of more than one enzyme complex from the same original sample aliquot in chemoplexed analysis, the multiplexing of analysis of enzyme complexes.

### EXAMPLES

### Example 1

Substrate, enzyme and product were measured in a single sample from incubation of the drug Phenacetin as a substrate with human liver microsomes. Acetaminophen is widely used as a selective metabolite marker for the P450 1A2 enzyme. Enzyme activity for P450 1A2 in human liver microsomes using the drug Phenacetin as a substrate was performed resulting in acetaminophen as the reaction product. Three members of the enzyme complex—the substrate, the metabolite, and the enzyme were measured in a single experiment using a single sample in a concerted analytical process.

Methods. Enzyme activity was conducted using human liver microsomes from four different lots of microsome. The High, (Single Donor Lot HU8022 high activity), Low (Single Donor Lot HU8045 low activity) and HMM (50 Donor Pool, Lot PL050B) were purchased from Life Technologies Invitrogen. The BD microsomes were purchased from BD Gentest® (BD Supersomes Human CYP1A2, Lot 2354653).

Reaction incubations were performed using 0.5 mg/ml microsomal protein in 100 mM monobasic potassium phosphate, pH 7.4 containing 5 mM magnesium chloride. The substrate was added to a final concentration of 10 uM. The reactions were incubated at 37 degrees Celsius for times indicated on the results. Reactions were started by addition of NADPH (1 mM final concentration) after a five minute pre-incubation. The 100 uL reactions were quenched by addition of 10 mM DTT and heating to 95 degrees Celsius for 10 minutes. The sample was allowed to cool to room temperature and incubated for 30 minutes with iodoacetamide at room temperature in the dark. Trypsin was added to the sample to a final concentration of 50 mM and allowed to catalyze the cleavage of the proteins into peptides. This reaction took place at 37 degrees Celsius for 15 hours. The digestion was stopped by the addition of 10 uL of 10% formic acid in water. The resulting peptide substrate and metabolite mixture was analyzed by LC-MS as follows.

Liquid chromatography (HPLC) coupled to mass spectrometry (MS) was used to quantify the substrate, the product, and the enzyme from the sample work up. All measurements were made using an Eksigent® C18 column (available from Eksigent® Technologies, LLC of California, USA) with dimensions of 1.0 mm×50 mm packed with 3 um particles having a pore size of 300 Angstroms. HPLC separation was effected using 50 uL/min flow rates of a gradient elution with acetonitrile and water as the two mobile phase components. The gradient used for this example is listed in Table 1.

TABLE 1
HPLC Gradient

| | % B (0.1% formic |
|---|---|
| Time (minutes) | acid in Acetonitrile) |
| 0 | 5 |
| 0.5 | 5 |
| 2.5 | 45 |
| 3.0 | 85 |
| 3.6 | 85 |
| 4.1 | 5 |
| 5.0 | 5 |

Quantification of the substrate, enzyme and product was performed as commonly known in the art by using calibration curves generated by adding known amounts of each component to naïve matrix, which in this case was rat liver microsomes, to generate standard samples of known concentration. The standards were measured along with the samples according to the above procedure. The standards were used to determine the relationship between detector response and analyte concentration, a response factor which is the slope of the calibration curve. Sample concentrations were back-calculated using the measure detector response and the response factor.

Materials. All materials used were obtained from commercial sources. Three lots of liver microsomes were obtained from Invitrogen, part of Life Technologies Corp. (Durham, N.C.). The recombinant enzyme microsomes and one lot of human liver microsomes were purchased from BD Gentest®. Chemicals were purchased from Sigma-Aldrich® Co., LLC, including, monobasic potassium phosphate, NADPH, magnesium chloride, trypsin, Labetalol, Phenacetin and Acetaminophen. Peptide internal standards were purchased from AB Sciex (Foster City, Calif.) as part of the Human Induction Kit.

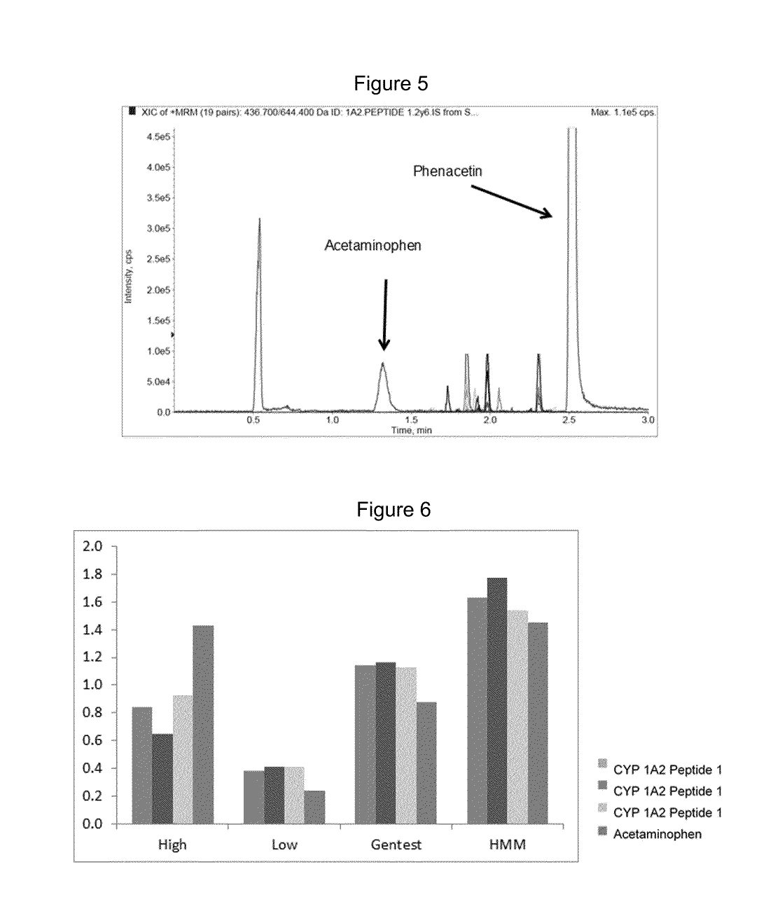

Results. The results of the experiment are shown in FIG. 5 which is a plot of detector response for each of the 1A2 peptides chosen to represent the amount of 1A2 enzyme in the sample and the product of the reaction, acetaminophen. The data show the variation in both formation of product and content of P450 1A2 enzyme for the four different lots of microsomes. The results are in agreement with the manufacturer's activity measurements and what might be expected for the associated enzyme content which was not measured by the manufacturer. FIG. 6 shows the related enzyme complex components analyzed by and in a single analytical event. The plot shows detector response for the various chemotypes versus retention time on the HPLC column for the chemoplexed assay. The data demonstrate the reduction to practice of the invention measuring the enzyme complex as a unit in a single analytical procedure.

FIG. 5 is a plot showing the detector response versus chromatographic retention time for each of the measured enzyme complex components from a single sample run in a concerted analytical method. The chemoplexed analysis permits the determination of the concentration of both small molecules and large molecules with a single analysis.

Shown in FIG. 6 is the mean normalized response for the peptides chosen to represent the P450 1A2 enzyme and the product of the reaction, acetaminophen which were determined in a chemoplexed analysis of the 1A2 enzyme complex from human liver microsomes.

### Example 2

Substrate, enzyme and product were measured in a single sample from incubation of the drug azidothymidine (AZT) as a substrate with recombinant UGT2B7 microsomes purchased from BD Biosciences. Enzyme activity for UGT2B7 in Supersomes using the drug azidothymidine (AZT) as a substrate was performed resulting in AZT-glucuronide product. All three members of the enzyme complex—the substrate, the metabolite, and the enzyme were measured.

Methods. Enzyme activity was conducted using increasing amounts of UGT2B7 recombinant protein from overexpression system microsomes in an experiment typically performed to examine enzyme kinetics. Reaction incubations were performed using 0.2 mg/ml microsomal protein in 100 mM monobasic potassium phosphate, pH 7.4 containing 5 mM magnesium chloride, 25 ug/ml alamethicin, 5 mM saccharic acid lactone and 2 mM UDPGA. The reactions were incubated at 37 degrees Celsius for 20 minutes. Reactions were started by addition of substrate after a five minute pre-incubation. The 100 uL reactions were quenched by addition of 200 uL of acetonitrile containing 0.1% formic acid and labetalol internal standard at 40 ng/ml. The quenched samples were centrifuged at 14000 rpm for ten minutes to pellet the microsomal protein. 50 uL of the supernatant was measured, diluted with 50 ul of water containing 0.1% formic acid, and analyzed by the LC-MS method described below. The protein pellet from each reaction was digested to generate peptides as follows. The remaining supernatant from the reaction incubation and protein precipitation was removed to a clean tube. The protein pellet in the original tube was washed with cold acetonitrile, re-suspended by addition of 90 uL of ammonium bicarbonate buffer at pH 7.6 and sonication for five minutes. The protein suspension was heated to 95 degrees Celsius for 10 minutes in the presence of 10 mM DTT. The protein suspension was allowed to cool to room temperature and incubated for 30 minutes with iodoacetamide at room temperature in the dark. Trypsin was added to the protein suspension to a final concentration of 50 mM and allowed to catalyze the cleavage of the proteins into peptides. This reaction took place at 37 degrees Celsius for 15 hours. The digestion was stopped by the addition of 10 uL of 10% formic acid in water. The resulting peptide mixture was analyzed by LC-MS as follows.

Liquid chromatography (HPLC) coupled to mass spectrometry (MS) was used to quantify the substrate, the product, and the enzyme from the sample work up. All measurements were made using an Eksigent® C18 column (available from Eksigent® Technologies, LLC of California, USA) with dimensions of 1.0 mm×50 mm packed with 3 um particles having a pore size of 300 Angstroms. HPLC separation was effected using 50 uL/min flow rates of a gradient elution with acetonitrile and water as the two mobile phase components. The gradient used for this example is listed in Table 1.

TABLE 1
HPLC Gradient

| | % B (0.1% formic |
|---|---|
| Time (minutes) | acid in Acetonitrile) |
| 0 | 0 |
| 0.5 | 0 |
| 5.0 | 40 |
| 6.0 | 80 |
| 7.5 | 80 |
| 8.0 | 0 |
| 10.0 | 0 |

Quantification of the substrate, enzyme and product was performed as commonly known in the art by using calibration curves generated by adding known amounts of each component to naïve matrix, which in this case was rat liver microsomes, to generate standard samples of known concentration. The standards were measured along with the samples according to the above procedure. The standards were used to determine the relationship between detector response and analyte concentration, a response factor which is the slope of the calibration curve. Sample concentrations were back-calculated using the measure detector response and the response factor.

Materials. All materials used were obtained from commercial sources. The liver microsomes were obtained from Invitrogen, part of Life Technologies Corp. (Durham, N.C.). The recombinant enzyme microsomes were purchased from BD Gentest®. Chemicals were purchased from Sigma-Aldrich® Co., LLC. including, monobasic potassium phosphate, alamethicin, saccharic acid lactone, sucrose, magnesium chloride, trypsin, UDPGA, AZT, and AZT-glucuronide.

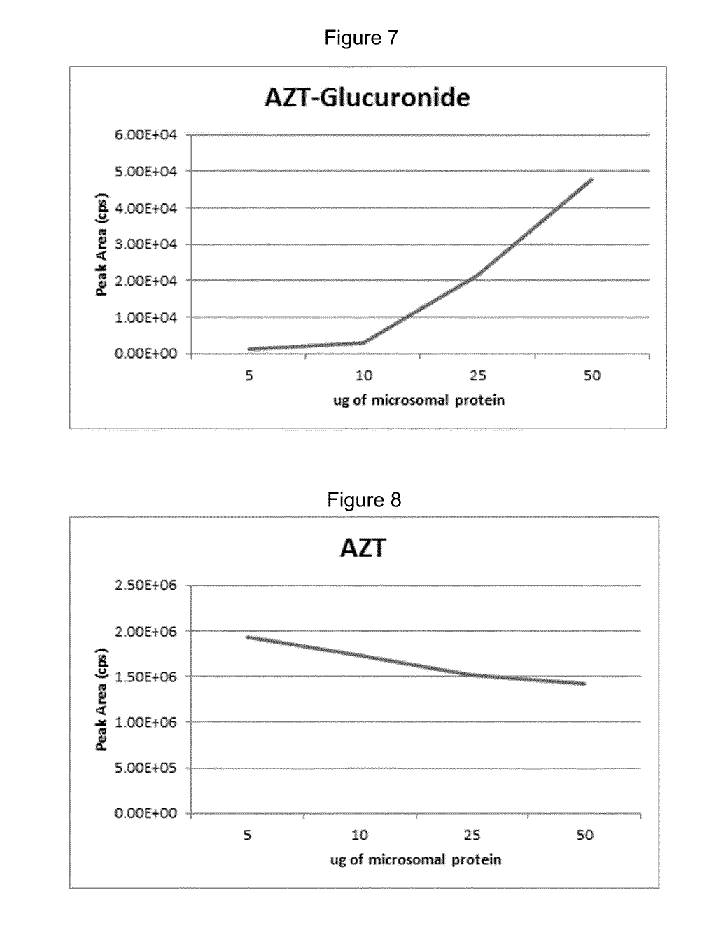

Results. The results of the experiment are shown in FIGS. 7-9 which are plots of detector response with added recombinant microsomal protein for the substrate AZT, the product AZT-glucuronide, and the enzyme UGT2B7. The data show the expected relationships between the enzyme complex components. As the added recombinant microsomal protein is increased, the amount of AZT remaining decreases, the amount of AZT-glucuronide increases and the amount of measured UGT2B7 increases. The data demonstrate the reduction to practice of the invention measuring the enzyme complex as a unit in a single analytical procedure.

FIG. 7 demonstrates the results showing the increasing formation of the enzyme complex product, AZT-glucuronide with increasing amount of recombinant microsomal protein added to an activity experiment.

FIG. 8 shows the results showing the consumption of the enzyme complex substrate, AZT, with increasing amount of recombinant microsomal protein added to an activity experiment.

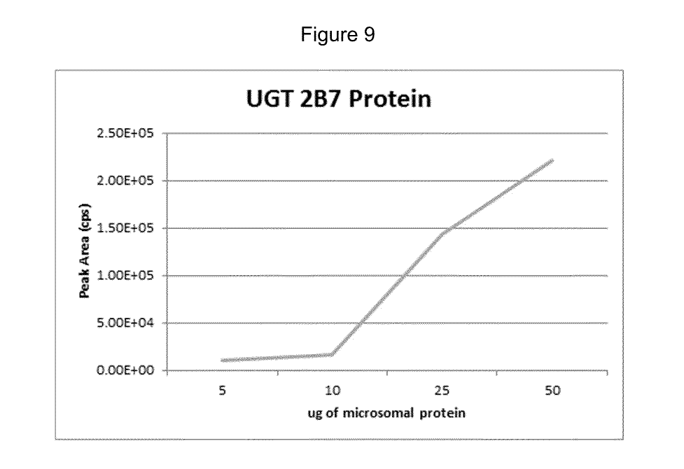

FIG. 9 is a graph of the results showing the increase in measured UGT2B7 protein found upon addition of recombinant microsomes over-expressing the UGT 2B7 protein to an enzyme activity experiment.

What is claimed is:

1. A method for quantifying the amount or concentration of small molecule and large molecule enzyme complex components derived from a biological matrix sample, the method comprising,
(a) digesting the large molecule proteins of the biological matrix sample into peptides using a chemical or enzymatic method,
(b) optionally, contacting the biological matrix sample with a solution having a pH less than 5.0 or greater than 8.0, an organic solvent or at least one affinity reagent to form a precipitate,
(c) optionally, separating the supernatant from the precipitate by centrifugation or filtration,
(d) optionally, treating the precipitate by resuspension in a solution having a pH in the range of 6.8 and 8.0, heating said solution to a temperature above 50° C., reducing said solution with at least one chemical reducing agent, and alkylating said solution,
(e) optionally, digesting the proteins present in the precipitate into peptides using a chemical or enzymatic method,
(f) determining the quantity of the enzyme complex related small molecules, and
(g) determining the quantity of the enzyme complex related peptides obtained in step (a) or step (e),

2. The method of claim 1 wherein the biological matrix is a natural fluid derived from an animal.

3. The method of claim 2 wherein the natural fluid is selected from blood, plasma, serum, cerebrospinal fluid, bile, or urine.

4. The method of claim 1 wherein the biological matrix is a natural fluid derived from a plant.

5. The method of claim 4 wherein the biological matrix fluid is selected from juice, sap, or oil.

6. The method of claim 1 wherein the biological matrix is selected from a tissue, a tissue homogenate, a cell, a cell lysate, or a fraction thereof.

7. The method of claim 1 wherein the enzyme complex comprises an enzyme, substrate, metabolite, inhibitor, activator, co-enzyme, co-factor and combinations thereof.

8. The method of claim 1 wherein the quantity of the enzyme complex component in step (a) or step (e) is determined by measuring the concentration or amount of peptides derived from the digested sample.

9. The method of claim 1 wherein the biological matrix sample is obtained from the same aliquot.

10. The method of claim 1 wherein the biological matrix sample is obtained from a mixture or combination of biological fluids or derivatives thereof.

11. The method of claim 1 wherein the enzyme complex components are measured in a concerted analytical workflow.

12. The method of claim 1, wherein said large molecules have a molecular weight of equal to or greater than 4000 Daltons.

13. The method of claim 1, wherein said small molecules have a molecular weight of less than 4000 Daltons.

14. The method of claim 1, wherein said small molecules are chemical elements of the Periodic Table.

15. The method of claim 1 wherein said large molecules are separated from the biological matrix by contact with a molecular weight cutoff membrane, size exclusion mechanism, centrifugation in a density gradient medium, solid phase extraction, or by affinity binding.

16. The method of claim 15 wherein the cutoff membrane effects separation of the molecules by dialysis, vacuum filtration, or centrifugation.

17. The method of claim 15 wherein the size exclusion mechanism is selected from gel filtration chromatography, gel electrophoresis, size exclusion chromatography and gel permeation chromatography.

18. The mechanism of claim 15 wherein the solid phase extraction is selected from ion exchange, reverse phase, mixed mode, or hydrophilic interaction liquid chromatography.

19. The method of claim 1 wherein the quantity of enzyme complex components is determined by liquid chromatography-atmospheric pressure ionization mass spectrometry, capillary electrophoresis-atmospheric pressure ionization mass spectrometry, direct introduction mass spectrometry, matrix assisted laser desorption ionization mass spectrometry, ion mobility, charged aerosol detection, radio-immunoassay, enzyme linked immunosorbent assay, a ligand binding technique and combinations thereof.
